(19) United States
(12) Patent Application Publication
(10) Pub. No.: US 2025/0214929 A1
(43) Pub. Date: Jul. 3, 2025

(54) SULPHONYL BENZENE DERIVATIVES AND METHODS FOR THE PREPARATION OF SULPHONYL BENZENE DERIVATIVES

(71) Applicant: HELMHOLTZ-ZENTRUM DRESDEN-ROSSENDORF E.V., Dresden (DE)

(72) Inventor: Austin CRAIG, Dresden (DE)

(73) Assignee: HELMHOLTZ-ZENTRUM DRESDEN-ROSSENDORF E.V., Dresden (DE)

(21) Appl. No.: 18/850,202

(22) PCT Filed: Apr. 26, 2023
(86) PCT No.: PCT/EP2023/060995

§ 371 Date: Sep. 24, 2024

(30) Foreign Application Priority Data
Apr. 28, 2022 (EP) 22170632.8

(51) Int. Cl. C07C 317/22; C07C 315/04; C07C 317/28; C07C 319/18; C07D 401/12; C07K 1/13; C07K 5/11; C07K 5/12; C07K 7/64

(52) U.S. Cl. CPC C07C 317/22; C07C 315/04; C07C 317/28; C07C 319/18; C07D 401/12; C07K 1/13; C07K 5/1019; C07K 5/12; C07K 7/64; C07B 2200/05

(57) ABSTRACT

Sulphonyl benzene derivatives, in particular to fluorinated sulphonyl benzene derivatives. A compound of formula I or general formula IA

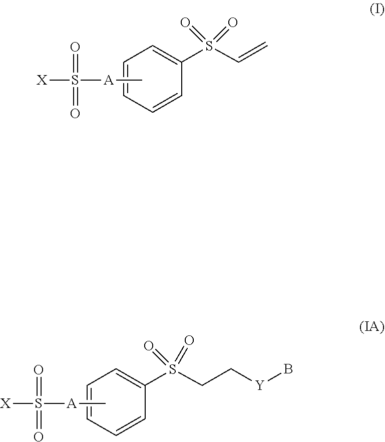

where X is fluorine; A is either O or NH; B is an amino acid- or peptide-containing group and Y is a secondary or tertiary amine group or a thioether group.

### FIELD

The invention relates to sulphonyl benzene derivatives, in particular to fluorinated sulphonyl benzene derivatives. Furthermore, it relates to methods for the preparation of sulphonyl benzene derivatives, in particular of fluorinated sulphonyl benzene derivatives. In addition, it relates to uses of sulphonyl benzene derivatives, in particular of fluorinated sulphonyl benzene derivatives.

### BACKGROUND

Positron emission tomography (PET), nowadays realized in the hybrid systems as a combination of positron emission tomography with computer tomography (PET/CT) or as a combination of positron emission tomography with magnetic resonance tomography (PET/MR), is a diagnostic non-invasive imaging modality used primarily in nuclear medicine for the real-time visualization of metabolic processes with high sensitivity and resolution.¹⁻⁶ Fluorine-18, the predominant radionuclide for PET imaging in the clinical setting, has advantageous physical characteristics including an optimal physical half-life (109.7 min), high positron branching (97% β⁺ decay), and low positron energy (0.635 MeV) making it ideal for molecular imaging of receptors, enzymes and transport systems and other metabolic processes in vivo. The predominant ¹⁸F-labeled radiotracer used in nuclear medicine is 2-deoxy-2-[¹⁸F]fluoro-D-glucose [[¹⁸F]FDG), which is used for the visualization of various cancers using PET.⁷ Aside from [¹⁸F]FDG, only a small number of 18F-labeled radiopharmaceuticals have been approved for routine patient applications in clinics.

The minority of clinically-applied radiotracers is largely due to lack of efficient available radiolabeling methods. This is mainly due to numerous practical limitations, as working with radionuclides necessitates that radiochemical reactions should proceed rapidly in order to account for product losses by radioactive decay. Additionally, the obligation to ensure safe production methods requires that radiosyntheses must proceed behind protective lead shielding (so called hotcells), in order to prevent radiation exposure to personnel. Therefore, the majority of clinical PET tracer preparations are carried out in automated radiosynthesizers installed in hotcells that are located in clean rooms. As a further obstacle, the transferability of standard fluorination chemistry methodology from organic synthesis is largely not applicable due to the different reactivity of fluorine (fluorine-18) on a picomolar and nonstoichiometric scale. Therefore, efficient radiofluorination methods towards radiotracer preparations are highly sought after.

To date, there have been numerous radiofluorination methods described, with the more recent findings focusing on “late-stage radiofluorination” protocols.⁸⁻²¹ Latestage radiofluorination methods incorporate the fluorine-18 radionuclide in the final steps of a radiopharmaceutical preparation as this advantageously reduces the product losses from radioactive decay. For the majority of late-stage radiofluorination protocols, high reaction temperatures, the presence of metal-containing compounds and organic solvents are required in order to achieve fast radiofluorination rates. However, for the ¹⁸F-labeling of sensitive biomolecules such as peptides, proteins or antibodies, the incompatible conditions of late-stage radiofluorination methods result in product degradation or the unwanted generation of multiple radiolabeled products which cannot be easily separated in a timely manner.²²

In order to overcome these limitations, the use of ¹⁸F-labeled prosthetic groups has been considered. Prosthetic groups typically contain both a functional group suitable for radiofluorination, and a functional group used for coupling to a biomolecule.²³ Coupling of the prosthetic groups to the biomolecule is also referred to as bioconjugation. FIG. 1 illustrates such a bioconjugation with a schematic example. Here, prosthetic group 2 binds to a biomolecule 1 whereby a conjugate 3 is obtained. For this purpose, biomolecule 1 has a functional group 1A to which a functional group 2A of the prosthetic group 2 can bind. Additionally, prosthetic group 2 has a second functional group suitable for radiofluorination. In the example shown in FIG. 1 the prosthetic group 2 already has a radiofluorinated [¹⁸F]fluoro group 2B instead of said second functional group. Binding between the biomolecule 1 and the prosthetic group 2 is established via functional group 1A of biomolecule 1 and functional group 2A of prosthetic group 2.

Typically, prosthetic groups are molecules which can be radiofluorinated under harsh radiofluorination conditions, purified and then bioconjugated to the biologically active molecule of interest under mild conditions in a two-step radiosynthesis, thereby circumventing the aforementioned limitations. Maleimide analogues as shown in general formula PA-1

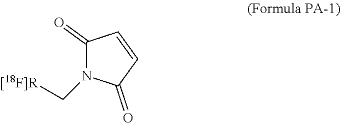

are early examples of highly chemoselective thiol-reactive prosthetic groups used for the ¹⁸F-labeling of biomolecules.²⁴⁻²⁷ However, the multi-step preparation of these prosthetic groups typically affords the compounds in (decay-corrected) radiochemical yields (RCY) of <20%, and often requires preparation times of 2 hours or more. Furthermore, the possible instability of the ¹⁸F-labeled products via retro-Michael reaction is a pertinent drawback of this approach.²⁸⁻³⁰ Examples of compounds of general formula PA-1 are [¹⁸F]FBAM (4-[¹⁸F]fluorobenzaldehyde-O-[6-(2,5-dioxo-2,5-dihydropyrrol-1-yl)-hexyl]oxime), [¹⁸F]FBOM (4-[¹⁸F]fluorobenzaldehyde-O-(2-{2-[2-(pyrrol-2,5-dione-1-yl) ethoxy]-ethoxy}-ethyl) oxime), and [¹⁸F]SFB (succinimidyl-4-[¹⁸F]fluorobenzoate). These examples exhibited radiochemical yields (RCY) of from 19-38% and molar activities Aₘ of from 35-76 GBq/μmol.

The advantages of vinyl sulfones towards the ¹⁸F-labeling of biomolecules demonstrated the rapid and chemoselective bioconjugation of [¹⁸F]F-DEG-VS (see Formula PA-2) to various peptides including c(RGDyC), used for tumor imaging using PET.³¹

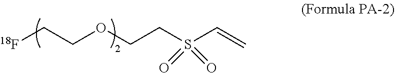

Additionally, the resulting PET tracers were found to be stable in vivo under various conditions compared to previous prosthetic groups. However, the requirement for time-consuming azeotropic drying, high precursor loadings (10 mg) and semi-preparative HPLC purification are considerable drawbacks of this approach. [¹⁸F]F-DEG-VS was obtained with a radiochemical yield (RCY) of from 35±6% and a molar activity Aₘ of from 19.2±4.3 TBq/mmol.

Gouverneur et al. have developed a thiol-selective prosthetic group towards the ¹⁸F-labeling of amino acids/peptides using a trifluoromethylation synthon (Scheme S-1).³²

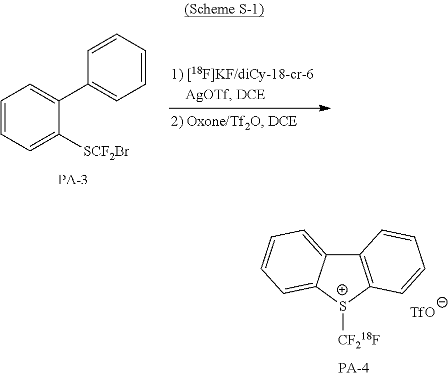

Here, compound PA-3 in a first step was reacted with [¹⁸F]KF and silver trifluoromethanesulfonate (AgOTf) in dichloroethane (DCE) for 20 min at 60° C. in the presence of a phase transfer catalyst. Subsequently the intermediate product in a second step was reacted with potassium peroxymonosulfate (Oxone®) and trifluoromethane sulphonic anhydride (Tf₂O) in DCE for 20 min at 45° C., thereby obtaining compound PA-4. Compound PA-4 was obtained with an activity yield (AY) of 5±2% (n=41), a radiochemical purity of >95%, and a molar activity Aₘ of 0.008 GBq/μmol.

Promisingly, the prosthetic group PA-3 was successfully applied to the radio-fluorination of c(RGDfC), a PSMA (prostate-specific membrane antigen) targeting ligand and an amyloid-β fragment. The latter two biomolecules being of significant importance for both the diagnosis of prostate cancer and Alzheimer's disease using PET. Despite the advances of this work, the preparation of the prosthetic group necessitated time-consuming multi-step preparation, a low Aₘ (molar activity) of 0.08 GBq/μmol and low activity yield (AY) (non-decay corrected isolated product yield).

A more recent example of a vinyl sulfone prosthetic group was described by Murphy et al., whereby the ¹⁸F-labeled prosthetic group is accessed via a uronium leaving group (Scheme S-2).³³

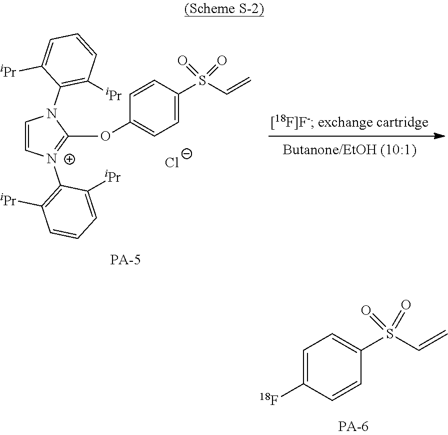 [SMILES: C=CS(=O)(=O)c1ccc(Oc2n(-c3c(C(C)C)cccc3C(C)C)cc[n+]2-c2c(C(C)C)cccc2C(C)C)cc1 ; [Cl-] ; C=CS(=O)(=O)c1ccc([18F])cc1]

Here, compound PA-5 is reacted to compound PA-6 with [¹⁸F]fluoride using an exchange cartridge in a mixture of butanone and ethanol (EtOH) (10:1) at 130° C. for 30 min. Compound PA-6 was obtained with a radiochemical yield (RCY) of 46±6%, a radiochemical purity of 85%, and a molar activity (Aₘ) of 106.2 GBq/μmol.

Advantageously, the positively charged prosthetic group PA-4 enabled the use of the compound to elute the [¹⁸F]⁻ F-bound on the QMA cartridge into the reaction vessel, thereby circumventing the need for azeotropic drying (i.e. exploiting the “minimalist approach”). Additionally, the prosthetic group was found to be obtained in good RCY of 46±4% in 43 min with a high Aₘ 106.2 GBq/μmol and was successfully used to ¹⁸F-labeling of various thiol-containing peptides including RGD analogues, a PSMA targeting derivative and a Bombesin analogue in good RCYs (55-93%). Despite the successful use of this prosthetic group the ¹⁸F-labeling of peptides, the disadvantages such as long reaction times, high temperatures, less <95% RCP and high precursor starting material (5-7 mg) limit their practical applications.

Wu et al. have highlighted an advantageous radiofluorination technique using aryl fluorosulfates via the ultra-fast sulfur [¹⁸F]fluoride exchange ([¹⁸F]SuFEx) reaction (Scheme S-3).³⁴

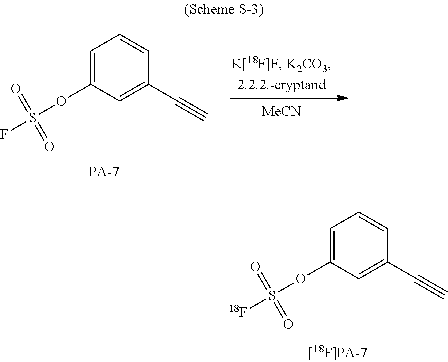

Here, 100 μg (0.5 μmol) of compound PA-7 were reacted with no carrier added (n.c.a.) [¹⁸F]fluoride which had an activity of ca. 3.7 GBq, in the presence of potassium carbonate and 2.2.2-cryptand in 0.5 mL of acetonitrile (MeCN) to compound [¹⁸F]PA-7. 0.5 mmol of compound [¹⁸F]PA-7 were obtained with a radiochemical yield (RCY_{HPLC}) of 99.3±0.6%.

In order to enable clinical use of radiofluorinated biomolecules it is desired to develop prosthetic groups containing both a functional group suitable for radiofluorination, and a functional group used for coupling to a biomolecule which on the one hand enable radiofluorination with high radiochemical yields and low reaction times and on the other hand permit binding to biomolecules under mild reaction conditions.

The problem of the invention is to eliminate the drawbacks according to the prior art. Thus, there is still the need to provide compounds having a fast-reacting and stable group for radiofluorination in addition to a functional group necessary for bioconjugation.

### SUMMARY

According to the invention there is provided a compound of general formula I or general formula IA

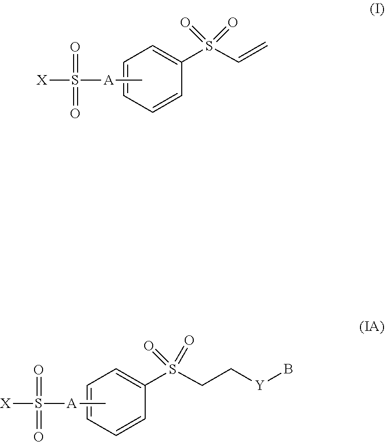

wherein
   X is fluorine;
   A is either O or NH;
   B is an amino acid- or peptide-containing group and
   Y is a secondary or tertiary amine group or a thioether group.

Compounds of general formula I can be used as precursors for the preparation of compounds of general formula IA. It may be provided to use compounds of general formula I, in which X is non-radioactive fluorine, for the preparation of compounds of general formula I, in which X is [¹⁸F]fluorine, as is shown in scheme 1:

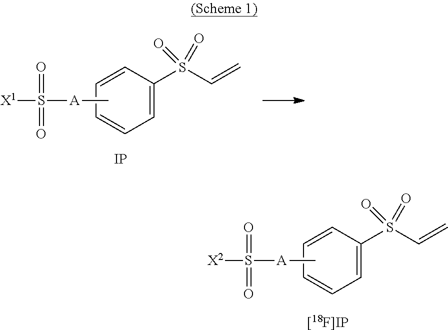

In the compound of general formula IP X¹ is non-radioactive fluorine. In the compound of general formula [¹⁸F]IP X² is [¹⁸F]fluorine. The compound of general formula [¹⁸F]IP differs from the compound of general formula IP only in that the compound of general formula [¹⁸F]IP has a [¹⁸F]fluorine group instead of a non-radioactive fluorine group. The compounds according to general formula [¹⁸F]IP are radiofluorinated sulfonyl benzene prosthetic groups. These compounds can be used for the preparation of ¹⁸F-labeled PET tracers.

In the scope of the present invention the term “non-radioactive fluorine” is to be understood as the non-radioactive isotope [¹⁹F]fluorine. The term “radioactive fluorine” is to be understood as the radioactive isotope [¹⁸F]fluorine.

The term “thioether group” means an-S-group.

The term “secondary amine group” means a group

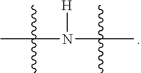 [SMILES: *N*]

The term “tertiary amine group” means a group

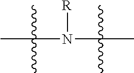

or a group

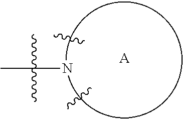

wherein R is a substituted or unsubstituted C₁-C₁₂ alkyl group and A is a cyclic or aromatic ring containing the nitrogen atom of the tertiary amine group. Ring A is part of group B. In this case, group B has a substituted or unsubstituted heteroaryl group with at least one nitrogen atom forming the tertiary amine group Y, or a substituted or unsubstituted heterocycloalkyl group with at least one nitrogen atom forming the tertiary amine group Y. An example of such a heterocycloalkyl group is a piperazine group.

The term “amino acid-containing group” means a group having a substituted or unsubstituted amino group and a substituted or unsubstituted carboxylic acid group. Examples of an “amino acid-containing group” are selected from the group consisting of cysteine or lysine.

The term “peptide-containing group” means a group having a peptide bond. For example, two or more amino acids can be bound to each other via one peptide bond.

The term “alkyl”, unless otherwise stated, in particular relates to a saturated aliphatic hydrocarbon group with a branched or unbranched carbon chain having 1 to 12 carbon atoms, preferably 1 to 8 carbon atoms, and especially preferred 1 to 6 carbon atoms. Examples of alkyl groups comprise, but are not limited to methyl, ethyl, propyl, isopropyl, isobutyl, sec-butyl, tert-butyl, pentyl, n-hexyl, octyl, dodecyl, and the like. Optionally, the alkyl group can be substituted with one or more substituents, with each substituent independently being hydroxy, alkyl, alkoxy, aryl, halo, haloalkyl, cyano, nitro, amino, monoalkylamino, or di-alkylamino, unless specifically stated otherwise.

The term “alkoxy”, unless otherwise stated, in particular relates to a group of formula-OR, wherein R is an alkyl group, as defined herein. Examples of alkoxy components comprise, but are not limited to methoxy, ethoxy, isopropoxy, and the like. Optionally, the alkoxy group can be substituted with one or more substituents, with each substituent independently being hydroxy, alkyl, alkoxy, aryl, halo, haloalkyl, cyano, nitro, amino, monoalkylamino, or dialkylamino, unless specifically stated otherwise.

The term “aryl”, unless otherwise stated, relates to a cyclic, aromatic hydrocarbon group consisting of a mono-, bi- or tricyclic aromatic ring system having 5 to 10 ring atoms, preferably 5 or 6 ring atoms. Optionally, the aryl group can be a substituted aryl group. Examples of aryl groups comprise, but are not limited to phenyl, naphthyl, anthracenyl, naphthalenyl, phenanthryl, fluorenyl, indenyl, pentalenyl, azulenyl, oxydiphenyl, biphenyl, methylenediphenyl, aminodiphenyl, diphenylsulfidyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like. The term “substituted aryl group” in particular relates to an aryl group optionally independently substituted with one to four substituents, preferably one or two substituents selected from but not limited to hydroxy, alkyl, alkoxy, aryl, halo, haloalkyl, cyano, nitro, amino, monoalkylamino, or dialkylamino and the like.

The term “heteroaryl” particularly relates to a monocyclic, bicyclic, or tricyclic group with 5 to 18 ring atoms, wherein at least one aromatic ring contains a nitrogen atom and either no further heteroatom or one, two, or three ring heteroatoms selected from N, O, or S, wherein the remaining ring atoms are C. The heteroaryl group can optionally be substituted. Examples of heteroaryl groups are-optionally substituted-imidazolyl, oxazolyl, isoxazolyl, thiazolyl, isothiazolyl, oxadiazolyl, thiadiazolyl, pyrazinyl, pyridazinyl, thiophenyl, furanyl, pyranyl, pyridinyl, pyrrolyl, pyrazolyl, pyrimidyl, quinolinyl, isoquinolinyl, quinazolinyl, benzofuranyl, benzothiophenyl, benzothiopyranyl, benzimidazolyl, benzoxazolyl, benzooxadiazolyl, benzothiazolyl, benzothiadiazolyl, benzopyranyl, indolyl, isoindolyl, indazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, wherein the listing is not complete.

The term “heterocycloalkyl”, unless otherwise stated, relates to a saturated cyclic ring having 5 to 18 ring atoms, wherein at least one aromatic ring contains a nitrogen atom and either no further heteroatom or one, two, or three ring heteroatoms selected from N, O, or S, wherein the remaining ring atoms are C. Examples of heterocycloalkyl groups comprise, but are not limited to piperidinyl, piperazinyl, homopiperazinyl, azepinyl, pyrrolidinyl, pyrazolidinyl, imidazolinyl, imidazolidinyl, oxazolidinyl, isoxazolidinyl, morpholinyl, thiazolidinyl, isothiazolidinyl, chinuclidinyl, thiadiazolylidinyl, benzothiazolidinyl, benzoazolylidinyl, dihydrofuryl, tetrahydrofuryl, dihydropyranyl, tetrahydropyranyl, thiomorpholinyl, thiomorpholinylsulfoxid, thiomorpholinylsulfon, dihydroquinolinyl, dihydroisoquinolinyl, tetrahydroquinolinyl, tetrahydroisoquinolinyl, 1-oxo-thiomorpholine, 1,1-dioxo-thiomorpholine, 1,4-diazepane, and 1,4-oxazepane. Optionally, the cycloalkyl group can be substituted with one or more substituents, with each substituent independently being hydroxy, alkyl, alkoxy, halo, haloalkyl, amino, monoalkylamino, or dialkylamino, unless specifically stated otherwise. Examples of cycloalkyl components comprise, but are not limited to cyclopropyl, cyclobutyl, cyclopentyl, cyclohexyl, cycloheptyl, and the like, including partially unsaturated derivatives thereof such as cyclohexenyl, cyclopentenyl, and the like.

The term “halo” relates to fluorine, chlorine, bromine, or iodine. It may be provided that the term “halo” only relates to chlorine, bromine, or iodine.

It may be provided that the compound of general formula [¹⁸F]IP is used for the preparation of a compound of general formula [¹⁸F]IA, as is shown in scheme 2:

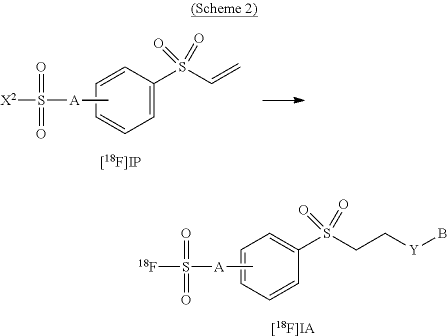

A compound of general formula [¹⁸F]IA is a compound of general formula IA, wherein X is [¹⁸F]fluorine and Y and B have the meanings given in connection with general formula IA.

A compound of general formula IP can be used as a prosthetic group, because it contains both a functional group suitable for radiofluorination, and a functional group used for coupling to a biomolecule. The functional group suitable for radiofluorination is the group -A-S(O)₂—X¹. The functional group used for coupling to a biomolecule is the group —S(O)₂—CH═CH₂ also referred to as a vinyl sulphonyl group. The group —S(O)₂—CH═CH₂ enables conjugation with a biomolecule.

Instead of the group -A-S(O)₂—X¹ a compound of general formula [¹⁸F]IP has -A-S(O)₂—X², wherein X² is [¹⁸F]fluorine. The group -A-S(O)₂—X² enables the use of the compound of general formula [¹⁸F]IP in an ultra-fast sulfur [¹⁸F]fluoride exchange ([¹⁸F]SuFEx) reaction, as is described by Wu et al.³⁴. The group —S(O)₂—CH═CH₂ enables conjugation with a biomolecule. Thus, the present invention provides a prosthetic group that is particularly suitable for the ¹⁸F-labeling of biomolecules due to the structural fusion of the groups -A-S(O)₂—X² and —S(O)₂—CH═CH₂.

A compound of general formula IP enables radiofluorination with high radiochemical yields and reduced reaction times. The resulting radiofluorinated compound, i.e. a compound of general formula [¹⁸F]IP, can bind to biomolecules under mild reaction conditions obtaining a conjugate, as shown in FIG. 1.

A compound of general formula IP can be used for the preparation of a compound of general formula IA, wherein X is non-radioactive fluorine. The method may be a one-stage method. For example, the method can be carried out like the method shown in scheme 2.

A preferred embodiment of the invention relates to a compound of general formula IP

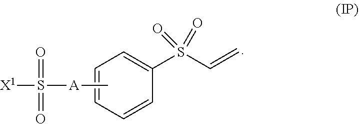

A compound of general formula IP is a compound of general formula I, wherein X is X¹, A is O or NH, and X¹ is non-radioactive isotope [¹⁹F]fluorine. The compounds of general formula IP can be used as precursors for the compounds of general formula IA. Particularly preferred is a compound of general formula IP, wherein group -A-S(O)₂—X¹ is in the para position to the vinyl sulphonyl group —S(O)₂—CH═CH₂.

In a preferred embodiment of the invention there is provided a compound of general formula IP-1

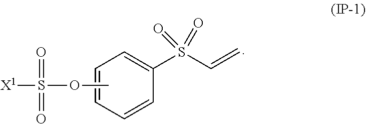

The compound of formula (IP-1) is a compound of general formula IP (I-P), wherein X¹ is non-radioactive fluorine and A is O. Particularly preferred is a compound of general formula IP-1, wherein the oxysulfonyl group —O—S(O)₂—X¹ is in the para position to the vinyl sulfonyl group —S(O)₂—CH═CH₂.

A preferred example of a compound of general formula IP-1 is 4-(vinylsulfonyl)phenyl sulfurofluoridate (VSPFS)

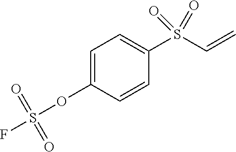

4-(Vinylsulfonyl)phenyl sulfurofluoridate is a compound of general formula I, wherein X is X¹, A is O, X¹ is non-radioactive fluorine, and the oxysulfonylgroup —O—S(O)₂—F is in the para position to the vinyl sulfonyl group —S(O)₂—CH═CH₂. Thus, specification “VSPFS” designates a compound, wherein fluorine is non-radioactive fluorine.

In a further preferred embodiment of the invention there is provided a compound of general formula IP-2

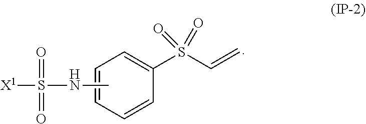

The compound of general formula IP-2 (is a compound of general formula IP, wherein X¹ is non-radioactive fluorine and A is NH). Particularly preferred is a compound of general formula IP, wherein the group —NH—S(O)₂—X¹ is in the para position to the vinyl sulfonyl group —S(O)₂—CH═CH₂.

A preferred example of a compound of general formula IP-2 is (4-(vinylsulfonyl)phenyl) sulfamoyl fluoride (VSPFSa)

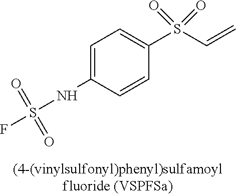

(4-(Vinylsulfonyl)phenyl) sulfamoyl fluoride is a compound of general formula I, wherein X is X¹, A is NH, X¹ is non-radioactive fluorine, and the sulfonamide group —NH—S(O)₂—F is in the para position to the vinyl sulfonyl group —S(O)₂—CH═CH₂. Thus, specification “VSPFSa” designates a compound, wherein fluorine is non-radioactive fluorine.

Another embodiment of the invention relates to a compound of general formula [¹⁸F]IP

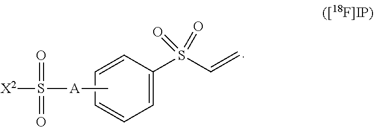

A compound of general formula [¹⁸F]IP is a compound of general formula I, wherein X is X², A is O or NH, and X² is [¹⁸F]fluorine. The compounds of general formula [¹⁸F]IP can be used as precursors for compounds of general formula IA, wherein X is [¹⁸F]fluorine. Particularly preferred is a compound of general formula [¹⁸F]IP, wherein the group —O—S(O)₂—X² is in the para position to the vinyl sulfonyl group —S(O)₂—CH═CH₂.

In a preferred embodiment of the invention there is provided a compound of general formula [¹⁸F]IP-1 ([¹⁸]F−)

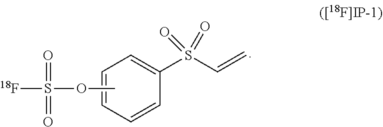

The compound of general formula [¹⁸F]IP-1 is a compound of general formula [¹⁸F]IP, wherein X² is [¹⁸F]fluorine and A is O. Particularly preferred is a compound of general formula [¹⁸F]IP-1, wherein the radiofluorinated sulfonic acid ester group —O—S(O)₂—X² is in the para position to the vinyl sulfonyl group —S(O)₂—CH═CH₂.

In a preferred embodiment of the invention there is provided a compound of general formula [¹⁸F]IP-2 ([¹⁸]F-I-2)

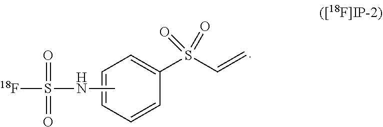

The compound of formula [¹⁸F]IP-2 is a compound of general formula ([¹⁸]FIP), wherein X² is [¹⁸F]fluorine and A is NH. Particularly preferred is a compound of general formula [¹⁸F]IP-2, wherein group —NH—S(O)₂—X¹ is in the para position to the vinyl sulfonyl group —S(O)₂—CH═CH₂.

In a more preferred embodiment of the invention there is provided the compound

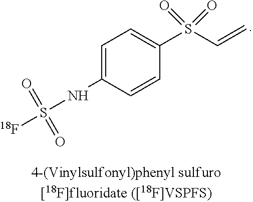 [SMILES: C=CS(=O)(=O)c1ccc(NS(=O)(=O)[18F])cc1]

4-(Vinylsulfonyl)phenyl sulfuro [¹⁸F]fluoridate is a compound of general formula I, wherein X is X², A is O, X² is [¹⁸F]fluorine, and the radiofluorinated oxysulfony group —O—S(O)₂—¹⁸F is in the para position to the vinyl sulfonyl group —S(O)₂—CH═CH₂. Thus, specification “[¹⁸F]VSPFS” designates a compound, wherein fluorine is [¹⁸F]fluorine.

In a more preferred embodiment of the invention there is provided the compound

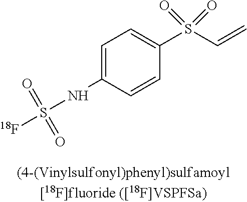 [SMILES: C=CS(=O)(=O)c1ccc(NS(=O)(=O)[18F])cc1]

(4-(Vinylsulfonyl)phenyl) sulfamoyl [¹⁸F]fluoride is a compound of general formula I, wherein X is X², A is NH, X² is [¹⁸F]fluorine, and the radiofluorinated sulfonamide group —NH—S(O)₂—¹⁸F is in the para position to the vinyl sulfonyl group —S(O)₂—CH═CH₂. Thus, specification “[¹⁸F]VSPFSa” designates a compound, wherein fluorine is [¹⁸F]fluorine.

In a further aspect of the invention there are provided a compound of general formula [¹⁸F]IA,

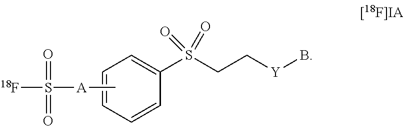

The compound of general formula [¹⁸F]1A is a compound of general formula IA wherein X is [¹⁸F]fluorine and A, B and Y have the meanings given in connection with general formula IA. Particularly preferred is a compound of general formula [¹⁸F]IA, wherein the oxysulfonyl group —O—S(O)₂—¹⁸F is in the para position to the group —S(O)₂—CH₂—CH₂—Y—B.

In a preferred embodiment of this aspect of the present invention there is provided a compound of general formula [¹⁸F]IA-1

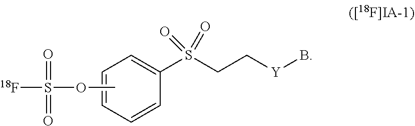

The compounds of formula [¹⁸F]IA-1 are compounds of general formula [¹⁸F]IA (I-A), wherein X is [¹⁸F]fluorine, A is O and Y and B have the meanings given in connection with formula (I-A). Particularly preferred is a compound of general formula [¹⁸F]IA-1, wherein the oxysulfonyl group —O—S(O)₂—¹⁸F is in the para position to the group —S(O)₂—CH₂—CH₂—Y—B.

In a more preferred embodiment of this aspect of the present invention there is provided a compound of general formula [¹⁸F]IA-1, wherein B is a peptide-containing group and Y is a secondary or tertiary amine group or a thioether group.

In a more preferred embodiment of this aspect of the present invention there is provided a compound of general formula [¹⁸F]IA-1, wherein B is a cysteine- or lysine-containing group and Y is a secondary or tertiary amine group or a thioether group.

In a still more preferred embodiment of this aspect of the present invention there is provided a compound of general formula [¹⁸F]IA-1, wherein B is a peptide-containing group and Y is a thioether group.

In a still more preferred embodiment of this aspect of the present invention there is provided a compound of general formula [¹⁸F]IA-2, wherein B is a cysteine- or lysine-containing group and Y is a thioether group.

Preferred examples of compounds of the general formula [¹⁸F]IA according to this aspect of the present invention are given in Table 1 below.

TABLE 1

| | Structure | Name |
|---|---|---|
| (i) | 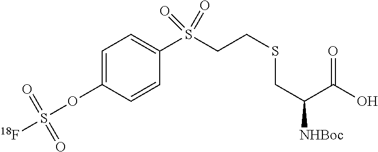 [SMILES: CC(C)(C)OC(=O)N[C@@H](CSCCS(=O)(=O)c1ccc(OS(=O)(=O)[18F])cc1)C(=O)O] | N-(tert-butoxycarbonyl)- S-(2-((4-(((fluoro- ¹⁸F)sulfonyl)oxy)phenyl) sulfonyl)ethyl)-L- cysteine |
| (ii) | 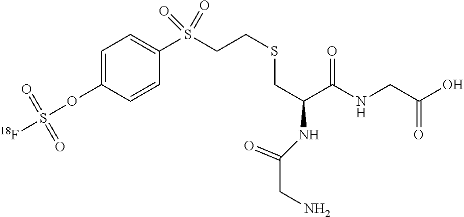 [SMILES: NCC(=O)N[C@@H](CSCCS(=O)(=O)c1ccc(OS(=O)(=O)[18F])cc1)C(=O)NCC(=O)O] | S-(2-((4-(((fluoro-¹⁸F) sulfonyl)oxy)phenyl) sulfonyl)ethyl)-N- glycyl-L-cysteinyl- glycine |
| (iii) | 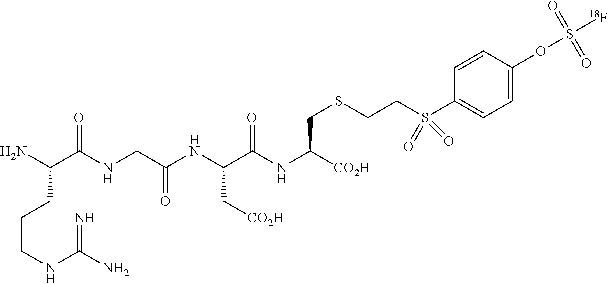 [SMILES: N=C(N)NCCC[C@H](N)C(=O)NCC(=O)N[C@@H](CC(=O)O)C(=O)N[C@@H](CSCCS(=O)(=O)c1ccc(OS(=O)(=O)[18F])cc1)C(=O)O] | (6S,12S,15R)-1,6- diamino-12-(carboxy- methyl)-15-(((2-((4- (((fluoro-¹⁸F)sulfonyl) oxy)phenyl)sulfonyl) ethyl)thio) methyl)-1-imino-7,10,13- trioxo-2,8,11,14- tetraazahexadecan- 16-oic acid |
| (iv) | 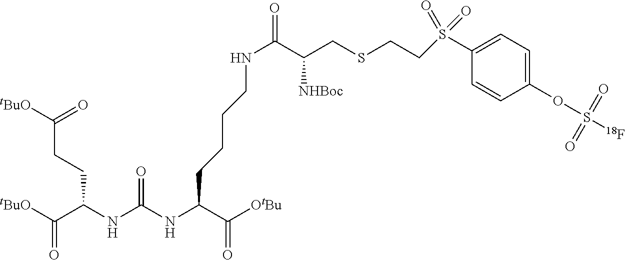 [SMILES: CC(C)(C)OC(=O)CC[C@H](NC(=O)N[C@@H](CCCCNC(=O)[C@H](CSCCS(=O)(=O)c1ccc(OS(=O)(=O)[18F])cc1)NC(=O)OC(C)(C)C)C(=O)OC(C)(C)C)C(=O)OC(C)(C)C] | tri-tert-butyl (6R, 13S,17S)-6-(((2-((4- (((fluoro-¹⁸F)sulfonyl) oxy)phenyl) sulfonyl)ethyl)thio) methyl)-2,2-dimethyl- 4,7,15-trioxo-3-oxa- 5,8,14,16-tetraazanon- adecane-13,17,19- tricarboxylate |
| (v) | 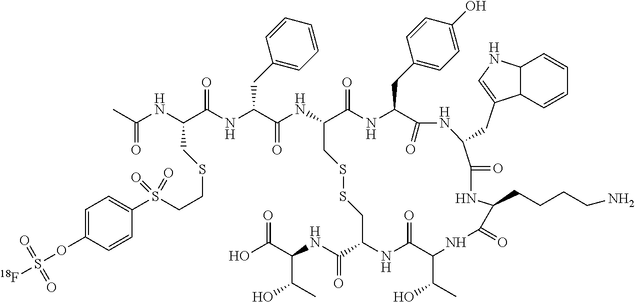 [SMILES: CC(=O)N[C@@H](CSCCS(=O)(=O)c1ccc(OS(=O)(=O)[18F])cc1)C(=O)N[C@H](Cc1ccccc1)C(=O)N[C@H]1CSSC[C@@H](C(=O)N[C@H](C(=O)O)[C@H](C)O)NC(=O)C([C@H](C)O)NC(=O)[C@H](CCCCN)NC(=O)[C@@H](CC2=CNC3C=CC=CC23)NC(=O)[C@H](Cc2ccc(O)cc2)NC1=O] | ((4R,10S,13R,16S,19R)- 19-((R)-2-((R)-2- acetamido-3-((2- ((4-(((fluoro-¹⁸F) sulfonyl)oxy)phenyl) sulfonyl)ethyl)thio) propanamido)-3- phenylpropanamido)- 10-(4- aminobutyl)-13-((3a, 7a-dihydro-1H-indol- 3-yl)methyl)-16-(4- hydroxybenzyl)-7-((S)- 1-hydroxyethyl)- 6,9,12,15,18- pentaoxo-1,2- dithia-5,8,11,14,17- pentaazacycloicosane-4- carbonyl)-L-allothreonine |
| (vi) | 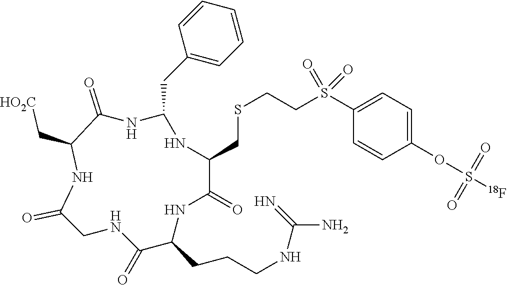 [SMILES: N=C(N)NCCC[C@@H]1NC(=O)[C@H](CSCCS(=O)(=O)c2ccc(OS(=O)(=O)[18F])cc2)N[C@@H](Cc2ccccc2)NC(=O)[C@H](CC(=O)O)NC(=O)CNC1=O] | 2-((2R,5S,11S,14R)- 2-benzyl-14-(((2-((4- (((fluoro-¹⁸F)sulfonyl) oxy)phenyl) sulfonyl)ethyl)thio) methyl)-11-(3- guanidinopropyl)- 4,7,10,13- tetraoxo-1,3,6,9,12- pentaazacyclotetradecan- 5-yl)acetic acid |

In another preferred embodiment of this aspect of the present invention there is provided a compound of general formula [¹⁸F]IA-1, wherein B is a peptide-containing group and Y is a secondary or tertiary amine group.

In another preferred embodiment of this aspect of the present invention there is provided a compound of general formula [¹⁸F]IA-1, wherein B is a peptide-containing group and Y is tertiary amine group.

In another preferred embodiment of this aspect of the present invention there is provided a compound of formula [¹⁸F]IA-1, wherein B is a lysine-containing group and Y is a secondary amine group.

Preferred examples of compounds of the general formula [¹⁸F]IA according to this aspect of the present invention are given in Table 2 below.

TABLE 2

| | Structure | Name |
|---|---|---|
| (vii) | 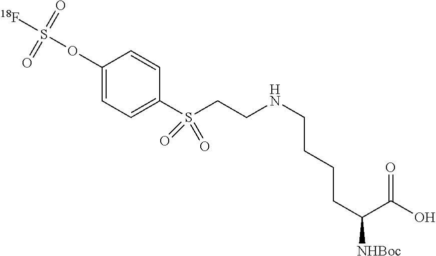 [SMILES: CC(C)(C)OC(=O)N[C@@H](CCCCNCCS(=O)(=O)c1ccc(OS(=O)(=O)[18F])cc1)C(=O)O] | N2-(tert-butoxycarbonyl)- N6-(2-((4- (((fluoro-¹⁸F)sulfonyl) oxy)phenyl) sulfonyl)ethyl)-L-lysine |
| (viii) | 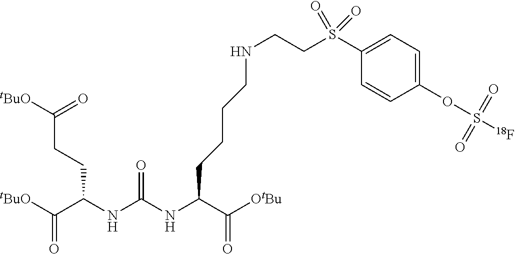 [SMILES: CC(C)(C)OC(=O)CC[C@H](NC(=O)N[C@@H](CCCCNCCS(=O)(=O)c1ccc(OS(=O)(=O)[18F])cc1)C(=O)OC(C)(C)C)C(=O)OC(C)(C)C] | di-tert-butyl (((S)-1- (tert-butoxy)-6-((2-((4- (((fluoro-¹⁸F)sulfonyl) oxy)phenyl) sulfonyl)ethyl)amino)- 1-oxohexan-2-yl) carbamoyl)-L-glutamate |
| (ix) | 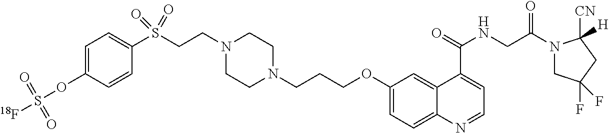 [SMILES: N#C[C@@H]1CC(F)(F)CN1C(=O)CNC(=O)c1ccnc2ccc(OCCCN3CCN(CCS(=O)(=O)c4ccc(OS(=O)(=O)[18F])cc4)CC3)cc12] | (S)-4-((2-(4-(3-((4-((2- (2-cyano-4,4-difluoro- pyrrolidin-1-yl)- 2-oxoethyl) carbamoyl)quinolin-6-yl) oxy)propyl)piperazin- 1-yl)ethyl)sulfonyl) phenyl sulfurofluoridate-¹⁸F |

In a further preferred embodiment of this aspect of the present invention there is provided a compound of general formula [¹⁸F]IA-2

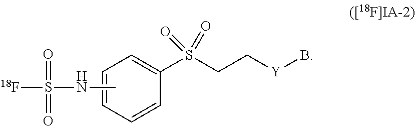

A compound of general formula [¹⁸F]IA-2 is a compound of general formula IA, wherein X is [¹⁸F]fluorine, A is NH and Y and B have the meanings given in connection with formula IA. Particularly preferred is a compound of general formula [¹⁸F]IA-2, wherein the group —NHO—S(O)₂—¹⁸F is in the para position to the group —S(O)₂—CH₂—CH₂—Y—B.

In a more preferred embodiment of this aspect of the present invention there is provided a compound of general formula [¹⁸F]IA-2, wherein B is a peptide-containing group and Y is a secondary or tertiary amine group or a thioether group.

In a still more preferred embodiment of this aspect of the present invention there are provided compounds of formula [¹⁸F]IA-2, wherein B is a peptide-containing group and Y is a thioether group.

Preferred examples of compounds of the general formula [¹⁸F]IA-2 according to this aspect of the present invention are given in Table 2a below.

TABLE 2a

| | Structure | Name |
|---|---|---|
| (ia) | 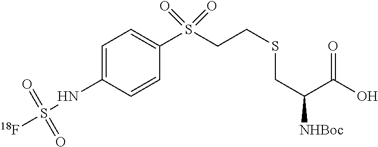 [SMILES: CC(C)(C)OC(=O)N[C@@H](CSCCS(=O)(=O)c1ccc(NS(=O)(=O)[18F])cc1)C(=O)O] | [¹⁸F]VSPFSa-conjugated N- Boc-Cys-OH |
| (iia) | 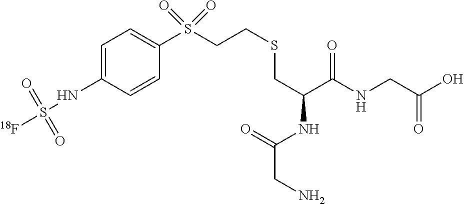 [SMILES: NCC(=O)N[C@@H](CSCCS(=O)(=O)c1ccc(NS(=O)(=O)[18F])cc1)C(=O)NCC(=O)O] | [¹⁸F]VSPFSa-conjugated H- Gly-Cys-Gly-OH |
| (iiia) | 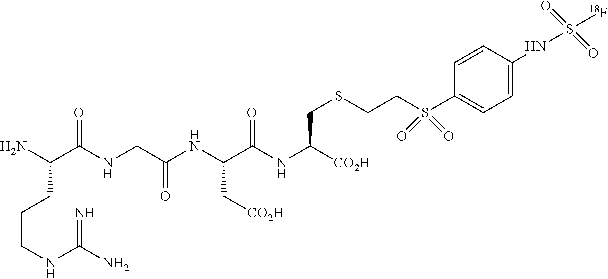 [SMILES: N=C(N)NCCC[C@H](N)C(=O)NCC(=O)N[C@@H](CC(=O)O)C(=O)N[C@@H](CSCCS(=O)(=O)c1ccc(NS(=O)(=O)[18F])cc1)C(=O)O] | [¹⁸F]VSPFSa-conjugated RGDC analogue |
| (iva) | 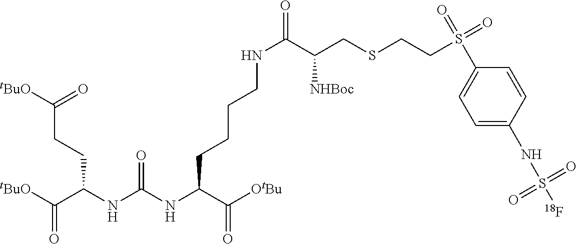 [SMILES: CC(C)(C)OC(=O)CC[C@H](NC(=O)N[C@@H](CCCCNC(=O)[C@H](CSCCS(=O)(=O)c1ccc(NS(=O)(=O)[18F])cc1)NC(=O)OC(C)(C)C)C(=O)OC(C)(C)C)C(=O)OC(C)(C)C] | [¹⁸F]VSPFSa-conjugated PSMA analogue |
| (va) | 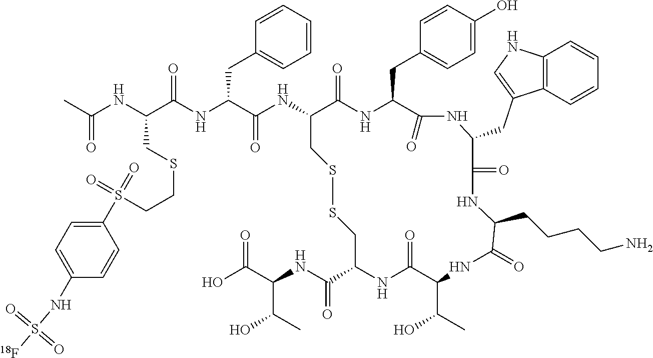 [SMILES: CC(=O)N[C@@H](CSCCS(=O)(=O)c1ccc(NS(=O)(=O)[18F])cc1)C(=O)N[C@H](Cc1ccccc1)C(=O)N[C@H]1CSSC[C@@H](C(=O)N[C@H](C(=O)O)[C@H](C)O)NC(=O)[C@H]([C@H](C)O)NC(=O)[C@H](CCCCN)NC(=O)[C@@H](Cc2c[nH]c3ccccc23)NC(=O)[C@H](Cc2ccc(O)cc2)NC1=O] | [¹⁸F]VSPFSa-conjugated TATE analogue |
| (via) | 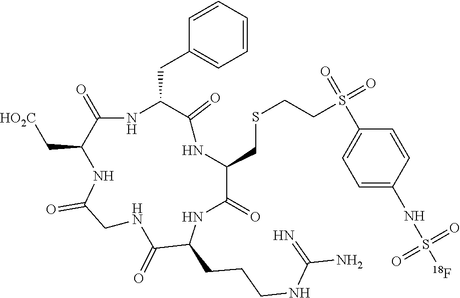 [SMILES: N=C(N)NCCC[C@@H]1NC(=O)[C@H](CSCCS(=O)(=O)c2ccc(NS(=O)(=O)[18F])cc2)NC(=O)[C@@H](Cc2ccccc2)NC(=O)[C@H](CC(=O)O)NC(=O)CNC1=O] | [¹⁸F]VSPFSa-conjugated cRGDC analogue |
| (viia) | 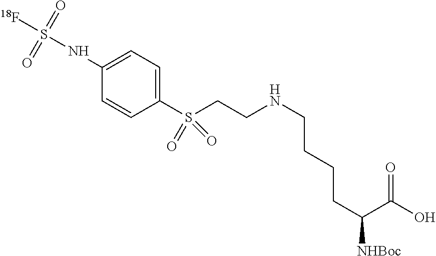 [SMILES: CC(C)(C)OC(=O)N[C@@H](CCCCNCCS(=O)(=O)c1ccc(NS(=O)(=O)[18F])cc1)C(=O)O] | [¹⁸F]VSPFSa-conjugated N- Boc-Lys-OH |
| (viiia) | 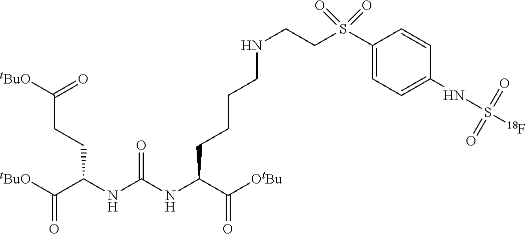 [SMILES: CC(C)(C)OC(=O)CC[C@H](NC(=O)N[C@@H](CCCCNCCS(=O)(=O)c1ccc(NS(=O)(=O)[18F])cc1)C(=O)OC(C)(C)C)C(=O)OC(C)(C)C] | [¹⁸F]VSPFSa-conjugated PSMA analogue |
| (ixa) | 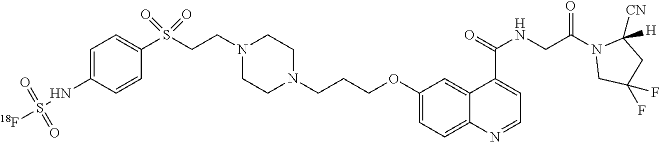 [SMILES: N#C[C@@H]1CC(F)(F)CN1C(=O)CNC(=O)c1ccnc2ccc(OCCCN3CCN(CCS(=O)(=O)c4ccc(NS(=O)(=O)[18F])cc4)CC3)cc12] | [¹⁸F]VSPFSa-conjugated FAPI analogue |

The compounds of formula [¹⁸F]IA-1 and [¹⁸F]IA-2 according to the invention have higher molar activities (Aₘ) compared to many of the known compounds, and thus are particularly suitable for routine clinical applications using positron-emission-tomography (in the present description often referred to as PET).

According to the invention additionally provided is a method for preparing a compound of general formula IP. For the preparation of a compound of general formula IP, a compound of general formula II

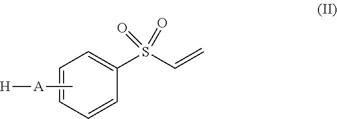

is reacted with a fluorosulfurylation agent. For example, the fluorosulfurylation can be done in two different ways: a) reaction of a compound of general formula II with 1,1′-sulfonyldiimidazole (SDI) at room temperature in an aprotic polar solvent via the ex situ generation of sulfuryl fluoride or b) reaction of a compound of general formula II with [4-(acetylamino)phenyl]imidodisulfuryl difluoride (AISF) at room temperature in an aprotic polar solvent.^{35,36}

A compound of general formula II can be obtained by chlorinating the compound of general formula III

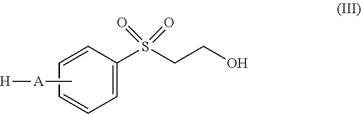

and subjecting the resulting compound an elimination reaction. The chlorination is carried out preferably with thionyl chloride and pyridine at room temperature in an aprotic polar solvent. The elimination reaction is done preferably with triethylamine at room temperature in an aprotic polar solvent, for example THF.

A compound of general formula III can be obtained by oxidizing the compound of formula IV

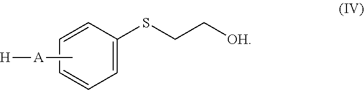

Preferably, the oxidation can be carried out by means of an oxidizing agent consisting of potassium peroxymonosulfate, potassium hydrogensulfate and potassium sulfate (Oxone®) at room temperature in a protic solvent. Preferably, the protic solvent is an organic protic solvent, more preferably an alcohol such as methanol (MeOH) or ethanol (EtOH), with MeOH being particularly preferred.

A compound of general formula IV, wherein A is O, can be obtained by reacting mercaptophenol with bromoethanol under basic conditions between 0° C. and room temperature. A compound of general formula IV, wherein A is NH, can be obtained by reacting (2,3,4)-aminothiophenol(s) with bromoethanol under basic conditions between 0° C. and room temperature.

In summary, Scheme 3 illustrates all steps of the method for preparing a compound of general formula IP.

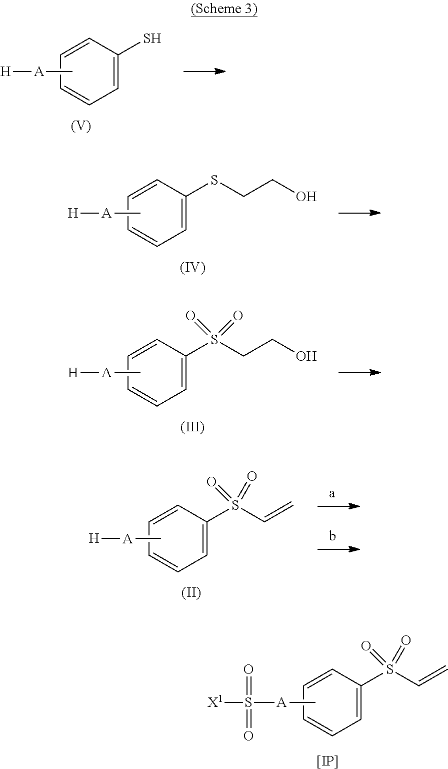

Arrows a and b indicate alternative methods for the conversion of a compound of general formula II to a compound of general formula IP. Arrow a indicates the reaction of a compound of general formula II with 1,1′-sulfonyldiimidazole (SDI) at room temperature in an aprotic polar solvent via the ex situ generation of sulfuryl fluoride. Arrow b indicates the reaction of a compound of general formula II with [4-(acetylamino)phenyl] imidodisulfuryl difluoride (AISF) at room temperature in an aprotic polar solvent. The method shown in scheme 3 can be carried out at ambient pressure.

Alternatively, a further method for the conversion of a compound of general formula II to a compound of general formula IP is to react a compound of general formula II with 1-(fluorosulfuryl)-2,3-dimethyl-1H-imidazol-3-ium trifluoromethansulfonate in an aprotic polar solvent at ambient pressure.

The method according to the invention is a facile synthesis for the preparation of a compound of general formula IP. The compound of general formula IP is a suitable precursor for radiofluorination. It enables the preparation of a compound of general formula [¹⁸F]IP (see scheme 1) under advantageous reaction conditions. In particular, the preparation can be done at room temperature in a short reaction time.

Scheme 4 illustrates the method according to the invention for preparing a compound of general formula IP using the example of the preparation of VSPFS. VSPFS contains no radioactive fluorine.

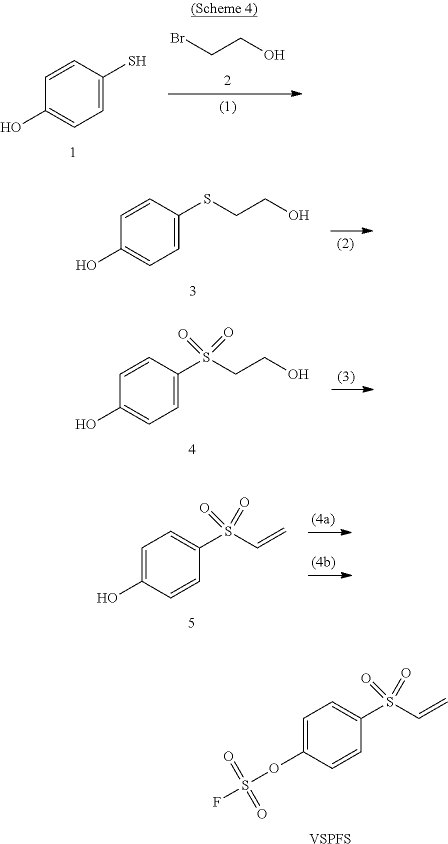

In step (1) mercaptophenol 1 is reacted with bromoethanol 2 to 4-((2-hydroxyethyl) thio) phenol 3. The reaction can take place in a methanolic solution containing sodium hydroxide solution. The reaction can take place at a temperature ranging between ° C. and room temperature. The reaction time can be 16 hrs. In step (2) 4-((2-hydroxyethyl) thio) phenol 3 is reacted with Oxone® to 4-((2-hydroxyethyl) sulfonyl) phenol 4. The reaction can be carried out in a protic solvent, preferably methanol (MeOH). The reaction can take place at room temperature. The reaction time can be 2 hrs. In step (3) 4-((2-hydroxyethyl) sulfonyl) phenol 4 is reacted to 4-(vinylsulfonyl) phenol 5. The reaction can take place in two stages. First, 4-((2-hydroxyethyl) sulfonyl) phenol 4 is chlorinated. Chlorination can be carried out with a chlorinating agent such as thionyl chloride (SOCl₂). Chlorination can be carried out in an aprotic solvent, such as methylene chloride (CH₂Cl₂). For example, it can be carried out in a mixture of pyridine and methylene chloride. In the mixture pyridine and methylene chloride can be present in a ratio of from 1:13.75 based on the volume of the mixture. The reaction can take place at room temperature. The reaction time can be 20 hrs. The intermediate obtained by chlorination subsequently is subjected to an elimination reaction to obtain 4-(vinylsulfonyl) phenol 5. Elimination can take place by means of an eliminant such as triethylamine (TEA). The eliminant is preferably a base. The reaction can take place in an aprotic polar solvent, preferably tetrahydrofurane (THF). The reaction can take place at room temperature. The reaction time can be 24 hrs. The fluorosulfurylation of 4-(vinylsulfonyl) phenol 5 to VSPFS can take place either according to step (4a) or step (4b). In step (4a) the reaction of 4-(vinylsulfonyl) phenol 5 can take place with sulfuryl fluoride formed in situ. In this instance, 4-(vinylsulfonyl) phenol 5 can be reacted with 1,1′-sulfonyldiimidazole (SDI) and KF, for example.³⁵ The reaction can take place in an aprotic polar solvent, for example in a mixture of TFA, HCOOH, TEA, and methylene chloride. The reaction can take place at room temperature. The reaction time can be 18 hrs. In step (4b) the reaction of 4-(vinylsulfonyl) phenol 5 to 4-(acetylamino)phenyl]imidodisulfuryl difluoride (AISF) can take place.³⁶ The reaction can take place in an aprotic polar solvent, for example in a mixture of 1,8-diazabicyclo [5.4.0]undec-7-ene (DBU) and tetrahydrofuran (THF) in a volume ratio of 1:10 The reaction can take place at room temperature. The reaction time can be 10 min.

Additionally, the reaction of 4-(vinylsulfonyl) phenol 5 with 1-(fluorosulfuryl)_{2,3}-dimethyl-1H-imidazol-3-ium trifluoromethansulfonate in dichloromethane at 0° C.-20° C. at ambient pressure can take place. The reaction time can be one hour.

Scheme 4a illustrates the method according to the invention for preparing a compound of general formula IP, wherein A is NH using the example of the preparation of VSPFSa. VSPFSa contains no radioactive fluorine.

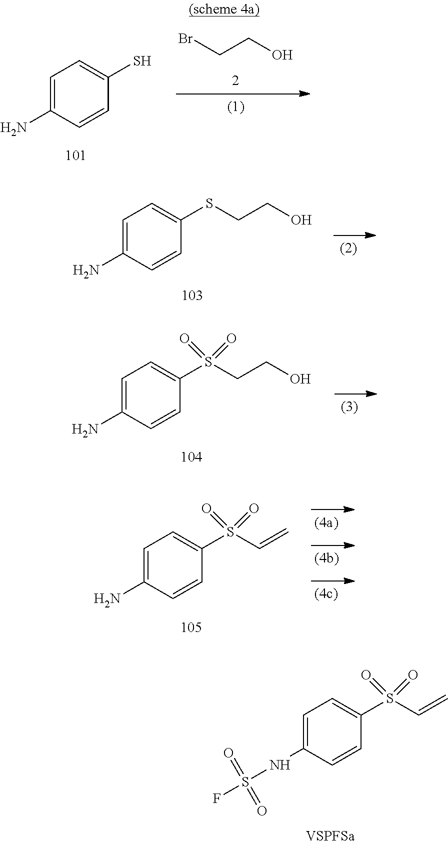

In step (1) 4-aminothiophenol 101 is reacted with bromoethanol 2 to 2-((4-aminophenyl) thio) ethanol 103. The reaction can take place in a methanolic solution containing sodium hydroxide solution. The reaction can take place at a temperature ranging between 0° C. and room temperature. The reaction time can be 16 hrs. In step (2) 2-((4-aminophenyl) thio) ethanol 103 is reacted with Oxone® to 2-(4-aminophenylsulfonyl) ethanol 104. The reaction can be carried out in a protic solvent, preferably methanol (MeOH). The reaction can take place at room temperature. The reaction time can be 2 hrs. In step (3) 2-(4-aminophenylsulfonyl) ethanol 104 is reacted to paminophenyl vinyl sulfone 105. The reaction can take place in two stages. First, 2-(4-aminophenylsulfonyl) ethanol 104 is chlorinated. Chlorination can be carried out with a chlorinating agent such as thionyl chloride (SOCl₂). Chlorination can be carried out in an aprotic solvent, such as methylene chloride (CH₂Cl₂). For example, it can be carried out in a mixture of pyridine and methylene chloride. In the mixture pyridine and methylene chloride can be present in a ratio of from 1:13.75 based on the volume of the mixture. The reaction can take place at room temperature. The reaction time can be 20 hrs. The intermediate obtained by chlorination subsequently is subjected to an elimination reaction to obtain p-aminophenyl vinyl sulfone 105. Elimination can take place by means of an eliminant such as triethylamine (TEA). The eliminant is preferably a base. The reaction can take place in an aprotic polar solvent, preferably tetrahydrofurane (THF). The reaction can take place at room temperature. The reaction time can be 24 hrs. The fluorosulfurylation of p-aminophenyl vinyl sulfone 105 to VSPFSa can take place either according to step (4a) or step (4b). In step (4a) the reaction of p-aminophenyl vinyl sulfone 105 can take place with sulfuryl fluoride formed in situ. In this instance, p-aminophenyl vinyl sulfone 105 can be reacted with 1,1′-sulfonyldiimidazole (SDI) and KF, for example.³⁵ The reaction can take place in an aprotic polar solvent, for example in a mixture of TFA, HCOOH, TEA, and methylene chloride. The reaction can take place at room temperature. The reaction time can be 18 hrs. In step (4b) the reaction of p-aminophenyl vinyl sulfone 105 to 4-(acetylamino)phenyl]imidodisulfuryl difluoride (AISF) can take place.³⁶ The reaction can take place in an aprotic polar solvent, for example in a mixture of 1,8-diazabicyclo [5.4.0]undec-7-ene (DBU) and tetrahydrofuran (THF) in a volume ratio of 1:10 The reaction can take place at room temperature. The reaction time can be 10 min. Additionally, in step (4c) the reaction of p-aminophenyl vinyl sulfone 105 with 1-(fluorosulfuryl)-2,3-dimethyl-1H-imidazol-3-ium trifluoromethansulfonate in dichloromethane at 0° C.-20° C. can take place. The reaction time can be one hour.

According to the invention additionally provided is a method for preparing a compound of general formula ([¹⁸F]IP)

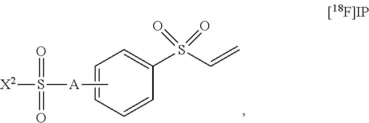

wherein X² is [¹⁸F]fluorine and A has the meanings given in connection with formula I. The method is shown in Scheme 1, above. The method comprises subjecting a compound of general formula I-P

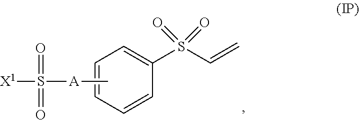

wherein X¹ is non-radioactive fluorine, a sulfur [¹⁸F]fluoride exchange reaction to obtain a compound of general formula [¹⁸F]I. Preferably, the sulfur [¹⁸F]fluoride exchange ([¹⁸F]SuFEx) reaction is done as ultra-fast [¹⁸F]fluoride exchange reaction in one step at room temperature for a very short time (0.5 to 1.0 min). The [¹⁸F]SuFEx reaction allows excellent ¹⁸F-fluorination rates and high radiochemical purity (RCP) above 95%. Thus, time-consuming HPLC purification of ¹⁸F-labeled products and metal compounds are avoided. Moreover, only small amounts of starting precursor per radiosynthesis are needed. In view of the half-life of [¹⁸F]fluorine it is particularly advantageous that the reaction of a compound of general formula IP to a compound of general formula [¹⁸F]IP can be carried out in a one-stage method.

Scheme 5 illustrates the method according to the invention for preparing a compound of general formula ([¹⁸F]IP) using the example of the preparation of [¹⁸F]VSPFS from VSPFS. The fluorine contained in VSPFS is non-radioactive fluorine.

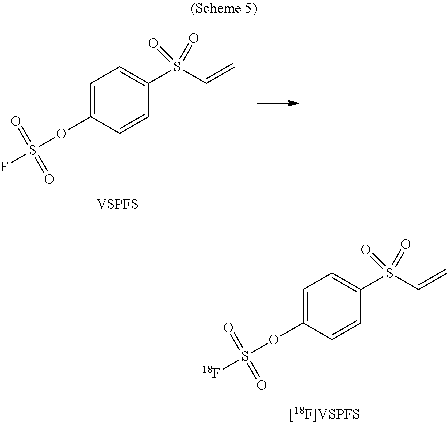

The reaction of VSPFS shown in scheme 5 to [¹⁸F]VSPFS is preferably carried out in an aprotic solvent, preferably acetonitrile (MeCN). The reaction can take place at room temperature (rt). The reaction time is short. For example, it may be 0.5 to 1 min. Radiofluorination can take place by means of a sulfur [¹⁸F]fluoride exchange reaction using tetrabutyl ammonium [¹⁸F]fluoride ([¹⁸F]TBAF).

According to the invention, additionally provided is a method for preparing compounds of general formula IA

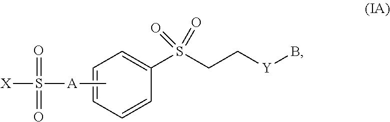

wherein X is [¹⁸F]fluorine and A and B have the meanings given in connection with general formula IA. The method is shown in scheme 2, above. The method comprises reacting a compound of formula [¹⁸F]IP with a compound of the formula H—Y—B, wherein Y and B have the meanings given in connection with formula IA.

It has been shown, that the vinyl sulfonyl group —S(O)₂—CH═CH₂ of the compound of general formula [¹⁸F]IP is particularly suitable for carrying out a bioconjugation. In particular, the vinyl sulfonyl group —S(O)₂—CH═CH₂ can be reacted with biomolecules having a thiol group, a primary amine group, and/or a secondary amine group. The reaction takes place under mild reaction conditions. Thus, the vinyl sulfonyl group —S(O)₂—CH═CH₂ enables binding of a compound of general formula [¹⁸F]IP to a biomolecule under mild reaction conditions. Mild reaction conditions are particularly meant to be a short reaction time and a reaction temperature that is at room temperature. Bioconjugation may be a chemoselective bioconjugation. Thus, functional group —S(O)₂—CH═CH₂ particularly allows a broad substrate scope for the chemoselective bioconjugation to, for example, thiols, primary and secondary amines. In particular, the group —S(O)₂—CH═CH₂ of [¹⁸F]VSPFS enables a broad substrate scope for the chemoselective bioconjugation to, for example, thiols, primary and secondary amines.

The compound of general formula H—Y—B may be a biomolecule, for example an amino acid, a peptide, or a protein. The compound of general formula H—Y—B can be reacted with the —S(O)₂—CH═CH₂ to obtain a biomolecule (see FIG. 1).

The compounds of general formula [¹⁸F]IA, in particular the compounds of general formula [¹⁸F]IA-1 and [¹⁸F]IA-2, have an excellent radiochemical purity (in the present description often referred to as RCP) and are available in high radiochemical yield (in the present description often referred to as RCY). Due to the fast-reacting and stable group for radiofluorination in addition to a functional group for bioconjugation they are particularly suitable for routine clinical applications using PET.

According to the invention, further there is provided the use of compounds of general formula [¹⁸F]IA, in particular the compounds of general formula [¹⁸F]IA-1 and [¹⁸F]IA-2 as radiopharmaceuticals or radiotracers for positron emission tomography.

In summary, the facile preparation of the compounds of general formula I enables the synthesis of the ¹⁸F-labeled compounds of formulas (i) to (ix) and numerous other ¹⁸F-labeled thiol- or amine-containing peptides and proteins in high activity yields, good molar activities and excellent radiochemical purity in a short time. One distinct advantage of this invention is the possibility to produce a variety of structurally diverse clinically-relevant ¹⁸F-labeled PET tracers using a commercially-available automated radiosynthesizer. The automation of this protocol greatly facilitates this PET tracer production method for routine patient use in clinics. Notably, the successful application of ¹⁸F-labeled compounds of formula I towards thiol- or amine-containing peptides and proteins such as PSMA inhibitor, RGD, TATE and FAPI analogues is a significant advantage of this ¹⁸F-labeling methodology over existing methods and highlights the versatility of this invention.

### BRIEF DESCRIPTION OF THE DRAWINGS

The invention is explained in detail with the help of examples. The drawings comprise FIG. 1 and FIG. 2, wherein

FIG. 1 is a scheme illustrating the bioconjugation between a prosthetic group and a biomolecule and

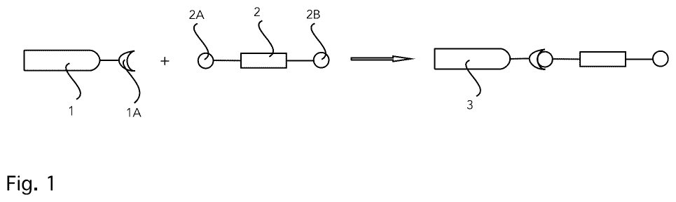

FIG. 2 shows in vitro binding assay results using [¹⁸F]VSPFS-TATE derivative in comparison with an established [⁶⁴Cu]Cu-Nodaga-TATE derivative.

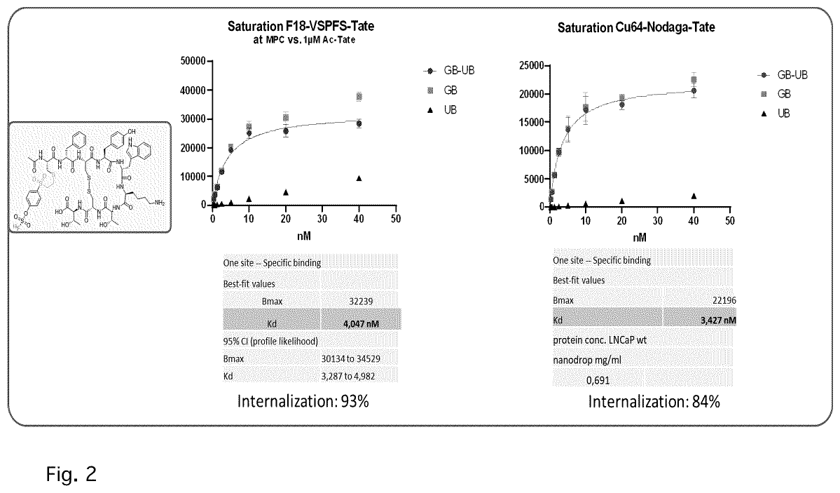

### DERAILED DESCRIPTION

### Example 1

### Preparation of 4-(vinylsulfonyl)phenyl fluorosulfate (VSPFS)

In a first step commercially available mercaptophenol 1 was reacted with bromoethanol 2 in NaOH and methanol between 0° C. and room temperature (rt) for 16 hrs. Thereafter, the reaction product 3 obtained in a yield of 64% was oxidized with Oxone® in MeOH at room temperature (rt) for 2 hrs (yield of 46%) to obtain compound 4. In the next step, the reaction product 4 was chlorinated with thionyl chloride in a solvent mixture of pyridine and methylene chloride in a ratio of from (13.75:1) CH₂Cl₂: pyridine based on the volume of the solvent mixture, at room temperature for 20 hrs and subsequently, an elimination reaction was performed with triethylamine (TEA) in tetrahydrofuran (THF) at room temperature for 24 hrs, to obtain compound 5 (yield 79%).

The target compound of this synthesis VSPFS was achieved in two different methods. Method 1: Reaction of the aryl vinyl sulfone compound 5 obtained in the last step via the ex situ generation of sulfuryl fluoride. Said reaction was carried out for 18 hrs in a fused two chamber reaction vessel where chambers A and B are connected by a small glass bridge to allow gaseous exchange between the chambers. Chamber A contained SDI, KF and HCOOH: TFA (0.9 mL HCOOH, 0.1 mL TFA), and chamber B contained TEA and 4 mL methylene chloride (with TEA concentration depending on the substrate concentration) (yield 38%). Method 2: Reaction of the aryl vinyl sulfone compound 5 obtained in the last step with commercially available solid 4-(acetylamino) phenyl]imidodisulfuryl difluoride (AISF) in DBU and THF at room temperature for 10 min (yield 42%).

### Example 2

### Preparation of 4-(vinylsulfonyl)phenyl [¹⁸F]fluorosulfate ([¹⁸F]VSPFS)

The following radiofluorination procedure was carried out: [¹⁸F]F⁻ was loaded onto a preconditioned QMA light carbonate cartridge which was subsequently eluted with tetraethylammonium bicarbonate (Et₄NHCO₃) (0.8 mg) in 0.7 mL MeOH, followed by evaporation of the solvent under reduced pressure at 70° C. for 5 min. Thereafter, the precursor VSPFS obtained in example 1 (0.1 mg, 3.7 mmol) in 0.5 mL MeCN was added to the cooled reaction vial, and allowed to react for [¹⁸F]SuFEx at room temperature for 1 min only and without stirring. The reaction was quenched by dilution with water (6 mL) followed by SPE-based purification using a preconditioned HLB cartridge. [¹⁸F]VSPFS was isolated by elution from the cartridge with MeOH/EtOH (1-3 mL).

[¹⁸F]VSPFS was obtained in an optimized manual radiosynthesis procedure with isolated activity yields (AY) of 39±7% (n=6) in 25 min. The implementation of the radiosynthetic protocol into a GE Tracerlab® automated radiosynthesizer afforded the target compound [¹⁸F]VSPFS in 20% AY, with >95% RCP and molar activity (Aₘ) of 40-50 GBq/mmol in approximately 30 min.

### Example 3

### Preparation of Compounds (i) to (vi)

For the bioconjugation, the [¹⁸F]VSPFS obtained in example 2 (50-500 MBq) was directly transferred into a reaction vial containing one of the thiol-containing compounds listed in Table 3 as (s-i)-(s-vi) (0.5-3.0 mg) in 1.1 mL MeOH/sodium borate buffer pH 8.5 (1:10) and reacted with 10-30 min stirring at 35° C. Radiochemical purity (RCP) and yield (RCY) were monitored by Radio-HPLC. Instead of MeOH another organic solvent may be used. A preferred solvent is MeOH or Ethanol (EtOH). Instead of sodium borate another buffer may be used. A preferred buffer is sodium borate or HEPES.

The radiochemical purity (RCP) was calculated by the division of the integrated product peak area by the total integrated ¹⁸F-labeled peaks. The identity of the ¹⁸F-labeled products was confirmed by coinjection with the “cold” ¹⁹F-reference standard. The radiochemical yield (RCY) was calculated by the multiplication of the RCP by the final activity by the starting activity.

TABLE 3

| | Structure | Name |
|---|---|---|
| (s-i) | 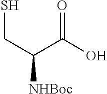 [SMILES: CC(C)(C)OC(=O)N[C@@H](CS)C(=O)O] | N-Boc-Cys-OH |
| (s-ii) | 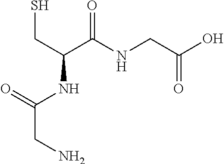 [SMILES: NCC(=O)N[C@@H](CS)C(=O)NCC(=O)O] | H-Gly-Cys-Gly-OH |
| (s-iii) | 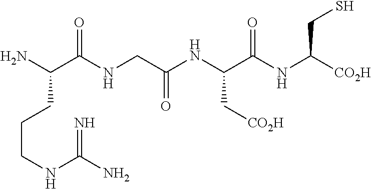 [SMILES: N=C(N)NCCC[C@H](N)C(=O)NCC(=O)N[C@@H](CC(=O)O)C(=O)N[C@@H](CS)C(=O)O] | RGDC |
| (s-iv) | 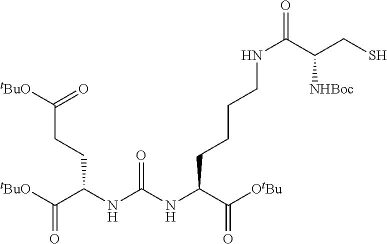 [SMILES: CC(C)(C)OC(=O)CC[C@H](NC(=O)N[C@@H](CCCCNC(=O)[C@H](CS)NC(=O)OC(C)(C)C)C(=O)OC(C)(C)C)C(=O)OC(C)(C)C] | PSMA analogue |
| (s-v) | 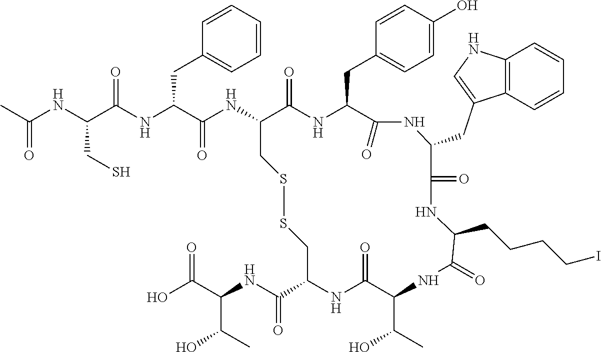 [SMILES: CC(=O)N[C@@H](CS)C(=O)N[C@H](Cc1ccccc1)C(=O)N[C@H]1CSSC[C@@H](C(=O)N[C@H](C(=O)O)[C@H](C)O)NC(=O)[C@H]([C@H](C)O)NC(=O)[C@H](CCCCI)NC(=O)[C@@H](Cc2c[nH]c3ccccc23)NC(=O)[C@H](Cc2ccc(O)cc2)NC1=O] | TATE analogue |
| (s-vi) | 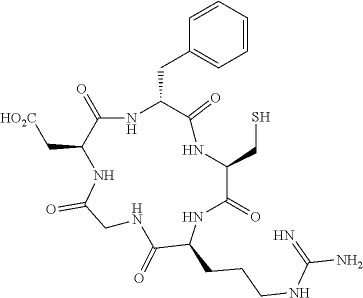 [SMILES: N=C(N)NCCC[C@@H]1NC(=O)[C@H](CS)NC(=O)[C@@H](Cc2ccccc2)NC(=O)[C@H](CC(=O)O)NC(=O)CNC1=O] | cRGDC analogue |
| (s-vii) | 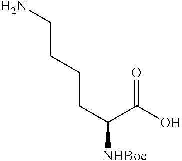 [SMILES: CC(C)(C)OC(=O)N[C@@H](CCCCN)C(=O)O] | N-Boc-Lys-OH |
| (s-viii) | 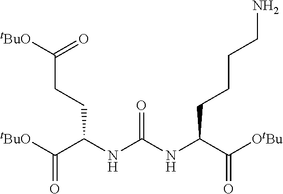 [SMILES: CC(C)(C)OC(=O)CC[C@H](NC(=O)N[C@@H](CCCCN)C(=O)OC(C)(C)C)C(=O)OC(C)(C)C] | (S)-di-tert-butyl 2-(3-((S)-6- amino-1-(tert-butoxy)-1- oxohexan-2-Y1)ureido) pentanedioate (PSMA analogue) |
| (s-ix) | 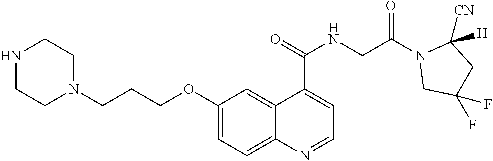 [SMILES: N#C[C@@H]1CC(F)(F)CN1C(=O)CNC(=O)c1ccnc2ccc(OCCCN3CCNCC3)cc12] | (S)-N-(2-(2-cyano-4,4-difluoro- pyrrolidin-1-yl)-2-oxoethyl)- 6-(3-(piperazine-1- yl)propoxy)quinoline-4- carboxamide (FAPI analogue) |

The radiochemical yield (RCY) and the radiochemical purity (RCP) of the resulted compounds (i) to (iii), (v) and (vi) are given in table 4.

### Example 4

### Preparation of Compounds vii to ix

Regioselective ¹⁸F-fluorination compounds via lysine/amine bioconjugation with [¹⁸F]VSPFS. Reaction conditions were identical to that of thiol-containing biomolecules. The corresponding amine-containing compounds used are listed in Table 3 as (s-vii)-(s-ix). The radiochemical yield (RCY_{HPLC}) of the resulted compounds (vii) to (ix) is given in table 4.

TABLE 4

| | | |
|---|---|---|
| | Radiochemical yield | Radiochemical purity |
| Compound | (RCY_{HPLC}) | (RCP) |

| | | | |
|---|---|---|---|
| (i) | 97% | (n = 3) | >95% |
| (ii) | 91 ± 4% | (n = 3) | >95% |
| (iii) | 97% | (n = 2) | >95% |
| (v) | 85 ± 4% | (n = 2) | >95% |
| (vi) | 97% | (n = 3) | >95% |
| (vii) | 29 ± 5% | (n = 2) | |
| (viii) | 30% | (n = 2) | |
| (ix) | >97% | (n = 2) | |

### List of Abbreviations

AISF [4-(acetylamino)phenyl]imido disulfuryl difluoride
SDI 1,1′-Sulfonyldiimidazole
Oxone® Potassium peroxysulfate
Et₃N Triethylamine—TEA
TBAF Tetrabutyl ammoniumfluoride
TFA Trifluoro acetic acid
MeOH Methanol
EtOH Ethanol
HEPES 4-(2-hydroxyethyl)-1-piperazineethanesulfonic acid
β⁺ positron
PSMA Prostate-specific membrane antigen
FAPI Fibroblast activated protein inhibitor
(c)RGDC (cyclic) Arginine-Glycine-Aspartate-Cysteine
TATE tyrosine3-octreotate ((Tyr3)-octreotate)
Lys lysine
Cys cysteine
Boc tert-butoxycarbonyl
RCY high radiochemical yield
RCP radiochemical purity
AY activity yield
Aₘ molar activity
n.c.a. no carrier added
SOCl₂ thionyl chloride
[¹⁸F] fluorine-18
[¹⁸F]F− [¹⁸F]fluoride
PET positron emission tomography
DBU 1,8-diazabicyclo [5.4.0]undec-7-ene
MR Magnetic resonance
CT computerized tomography

### REFERENCES

1. Fowler, J. S.; Wolf, A. P. Acc. Chem. Res. 1997, 30 (4), 181-188.
2. Phelps, M. E. Proc. Natl. Acad. Sci. U.S.A 2000, 97 (16), 9226-9233.
3. Gambhir, S. S. Nat. Rev. Cancer 2002, 2 (9), 683-693.
4. Tsien, R. Y. Nat. Rev. Mol. Cell Bio. 2003, 5516-5521.
5. Willmann, J. K.; van Bruggen, N.; Dinkelborg, L. M.; Gambhir, S. S. Nat. Rev. Drug Discovery 2008, 7 (7), 591-607.
6. Ametamey, S. M.; Honer, M.; Schubiger, P. A. Chem. Rev. 2008, 108 (5), 1501-1516.
7. Ido, T.; Wan, C. N.; Casella, V.; Fowler, J. S.; Wolf, A. P.; Reivich, M.; Kuhl, D. E. J. Labelled Compd. Radiopharm. 1978, 14 (2), 175-183.
8. Lee, E.; Kamlet, A. S.; Powers, D. C.; Neumann, C. N.; Boursalian, G. B.; Furuya, T.; Choi, D. C.; Hooker, J. M.; Ritter, T. Science 2011, 334 (6056), 639-642.
9. Huiban, M.; Tredwell, M.; Mizuta, S.; Wan, Z. H.; Zhang, X. M.; Collier, T. L.; Gouverneur, V.; Passchier, J. Nat. Chem. 2013, 5 (11), 941-944.
10. Graham, T. J. A.; Lambert, R. F.; Ploessl, K.; Kung, H. F.; Doyle, A. G. J. Am. Chem. Soc. 2014, 136 (14), 5291-5294.
11. Rotstein, B. H.; Stephenson, N. A.; Vasdev, N.; Liang, S. H. Nat. Commun. 2014, 5, 4365.
12. Sergeev, M. E.; Morgia, F.; Lazari, M.; Wang, C.; van Dam, R. M. J. Am. Chem. Soc. 2015, 137 (17), 5686-5694.
13. Neumann, C. N.; Hooker, J. M.; Ritter, T. Nature 2016, 534 (7624), 369-373.
14. Craig, A.; Kolks, N.; Urusova, E. A.; Zischler, J.; Brugger, M.; Endepols, H.; Neumauier, B.; Zlatopolskiy, B. Chem. Commun. 2020, 56, 9505-9508.
15. Chen, W.; Huang, Z.; Tay, N. E. S.; Giglio, B.; Wang, M.; Wang, H.; Wu, Z.; Nicewicz, D. A.; Li, Z. Science 2019, 364 (6446), 1170-1174.
16. Ichiishi, N.; Brooks, A. F.; Topczewski, J. J.; Rodnick, M. E.; Sanford, M. S.; Scott, P. J. H. Org. Lett. 2014, 16, 3224-3227.
17. Tredwell, M.; Preshlock, S. M.; Taylor, N. J.; Gruber, S.; Huiban, M; Passchier, J.; Mercier, J.; Génicot, C.; Gouverneur, V. Angew. Chem. Int. Ed. 2014, 53, 7751-7755.
18. Mossine, A. V.; Brooks, A. F.; Makaravage, K. J.; Miller, J. M.; Ichiishi, N.; Sanford, M. S.; Scott, P. J. H.; Org. Lett. 2015, 17, 5780-5783.
19. Makaravage, K. J.; Brooks, A. F.; Mossine, A. V.; Sanford, M. S.; Scott, P. J. H.; Org. Lett. 2015, 18 (20), 5440-5443.
20. Lee, S. J.; Makaravage, K. J.; Brooks, A. F.; Scott, P. J. H.; Sanford, M. S.; Angew. Chem. Int. Ed. 2019, 58, 3119.
21. Walter, N.; Betram, J.; Drewes, B.; bahutski, V.; Timmer, M.; Schütz, M. B.; Krämer, F.; Neumaier, F.; Endepols, H.; Neumaier, B.; Zlatopolskiy, B. Eur. J. Med. Chem. 2022, 237, 114383.
22. Schirrmacher, R.; Wängler, B.; Bailey, J.; Bernard-Gauthier, V.; Schirrmacher, E.; Wängler, C.; Seminars in Nuclear Medicine 2017, 47 (5), 474-492.
23. Chi, D. Y.; Kilbourn, M. R.; Katzenellenbogen, J. A.; Brodack, J. W.; Welch, M. J.; Int. J. Rad. Appl. Instrum. A. 1986, 37 (72), 1173-1180.
24. Koniev, O.; Wagner, A. Chem. Soc. Rev. 2015, 44, 5495-5551.
25. Baldwin, A. D.; Kiick, K. L. Bioconjugate Chem. 2011, 22, 1946-1953.
26. Shen, B.-Q.; Xu, K.; Liu, L.; Raab, H.; Bhakta, S.; Kenrick, M.; Parsons-Reponte, K. L.; Tien, J.; Yu, S.-F.; Mai, E.; Li, D.; Tibbitts, J.; Baudys, J.; Saad, O. M.; Scales, S. J.; McDonald, P. J.; Hass, P. E.; Eigenbrot, C.; Nguyen, T.; Solis, W. A.; Fuji, R. N.; Flagella, K. M.; Patel, D.; Spencer, S. D.; Khawli, L. A.; Ebens, A.; Wong, W. L.; Vandlen, R.; Kaur, S.; Sliwkowski, M. X.; Scheller, R. H.; Polakis, P.; Junutula, J. R. Nat. Biotechnol. 2012, 30, 184-189.
27. Adumeau, P.; Davydova, M.; Zeglis, B. M. Bioconjugate Chem. 2018, 29, 1364-1372.
28. de Bruin, B.; Kuhnast, B.; Hinnen, F.; Yaouancq, L.; Amessou, M.; Johannes, L.; Samson, A.; Boisgard, R.; Tavitian, B.; Dolle, F. Bioconjugate Chem. 2005, 16, 406-420.
29. Cai, W.; Zhang, X.; Wu, Y.; Chen, X. J. Nucl. Med. 2006, 47, 1172-1180.
30. Kiesewetter, D. O.; Jacobson, O.; Lang, L.; Chen, X. Appl. Radiat. Isot. 2011, 69, 410-414.
31. Wu, Z.; Li, L.; Liu, S.; Yakushijin, F.; Yakushijin, K.; Horne, D.; Conti, P. S.; Li, Z.; Kandeel, F.; Shively, J. E. J. Nucl. Med. 2014, 55, 1178-1184.
32. Verhoog, S.; Kee, C. W.; Wang, Y.; Khotavivattana, T.; Wilson, T. C.; Kersemans, V.; Smart, S.; Tredwell, M.; Davis, B. G.; Gouverneur, V. J. Am. Chem. Soc. 2018, 140, 1572-1575.
33. Ma, G.; McDaniel, J. W.; Murphy, J. M. Org. Lett. 2021, 23 (2), 530-534.
34. Zheng, Q.; Xu, H.; Wang, H.; Du, W.-G. H.; Wang, N.; Xiong, H.; Gu, Y.; Noodleman, L.; Sharpless, K. B.; Yang, G.; Wu, P. J. Am. Chem. Soc. 2021, 143 (10), 3753-3763.
35. Veryser, C.; Demaerel, J.; Bielinas, V.; Gilles, P.; De Borggraeve, W. M. Org. Lett. 2017, 19 (19), 5244-5247.
36. Zhou, H.; Mukherjee, P.; Liu, R.; Evrard, E.; Wang, D.; Humphrey, J. M.; Butler, T. W.; Hoth, L. R.; Sperry, J. B.; Sakata, S. K.; C. J. Helal, C. J.; am Ende, C. W. Org. Lett. 2018, 20 (3), 812-815.

1-15. (canceled)

16. A compound of formula I or general formula IA 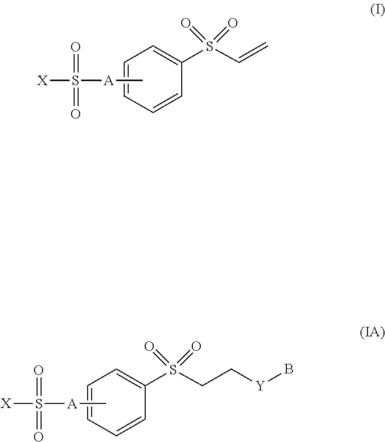
wherein
X is fluorine;
A is either O or NH;
B is an amino acid- or peptide-containing group and
Y is a secondary or tertiary amine group or a thioether group.

17. The compound according to claim 16, wherein the fluorine is [¹⁸F]fluorine.

18. The compound according to claim 16, wherein B is an amino acid-containing group, wherein the amino acid-containing group is a cysteine-containing group or a lysine-containing group.

19. The compound according to claim 16, wherein the compound of formula IA is selected from the group consisting of

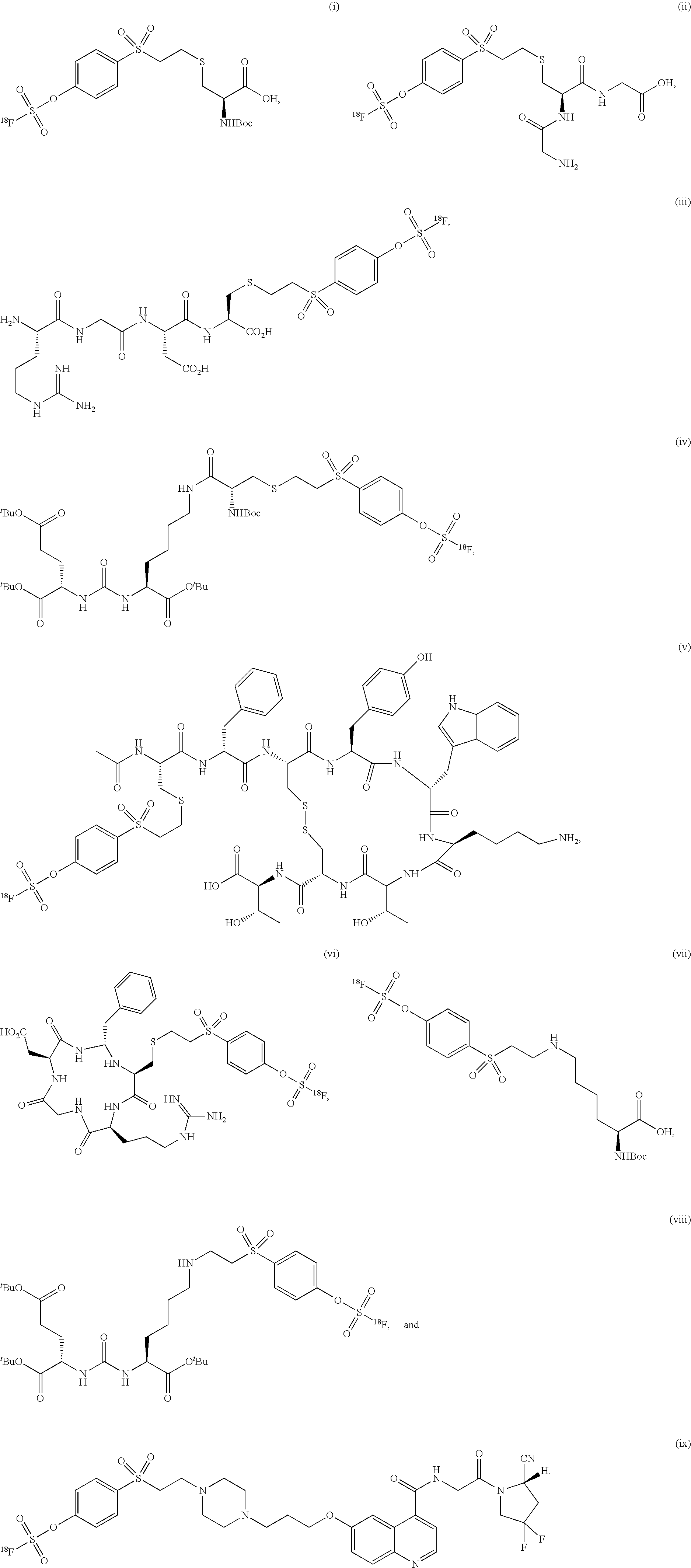

20. A method for preparing a compound of general formula IP 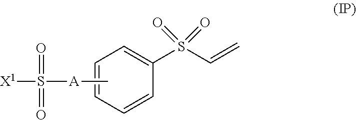
wherein X¹ is non-radioactive fluorine and A has the meanings given in claim 16, and reacting the compound of formula II 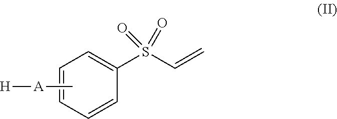
with a fluorosulfurylation agent.

21. The method according to claim 20, wherein the fluorosulfurylation agent is sulfuryl fluoride or [4-(acetylamino)phenyl]imido disulfuryl difluoride.

22. The method according to claim 20, wherein the compound of general formula II is obtained by chlorinating the compound of formula III 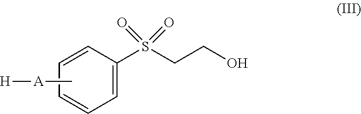
and subjecting the resulting compound an elimination reaction.

23. The method according to claim 22, wherein the compound of general formula III is obtained by oxidating the compound of formula IV

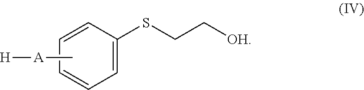

24. The method according to claim 23, wherein the oxidation is done by means of an oxidizing agent consisting of potassium peroxymonosulfate, potassium hydrogensulfate and potassium sulfate.

25. The method according to claim 23, wherein the compound of general formula IV is obtained by reacting mercaptophenol or aminothiophenol with bromoethanol.

26. A method for preparing compounds of general formula [¹⁸F]IP, 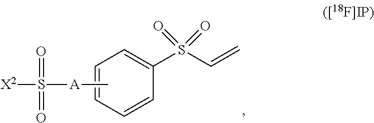
wherein X² is [¹⁸F]fluorine and A has the meanings given in claim 16, wherein a compound of general formula I-P 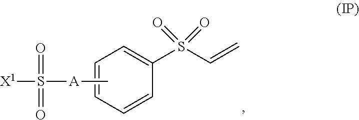
wherein X¹ is non-radioactive fluorine, is reacted to a compound of general formula [¹⁸F]I via a sulfur [¹⁸F]fluoride exchange reaction.

27. The method according to claim 26, wherein the exchange reaction is an ultra-fast sulfur [¹⁸F]fluoride exchange reaction ([¹⁸F]SuFEx).

28. A method for preparing a compound of general formula IA 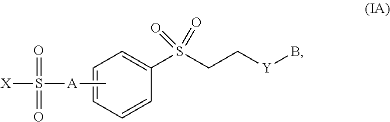
wherein X is [¹⁸F]fluorine and A has the meanings given in claim 16, and reacting a compound of formula [¹⁸F]IP with a compound of the formula H—Y—B.
